SEQUENCE LISTING (16 sequences)

SEQ ID NO: 1 - protein, 147 aa, Homo sapiens
MVMGLGVLLLVFVLGLGLTPPTLAQDNSRYTHFLTQHYDAKPQGRDDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRP

SEQ ID NO: 2 - protein, 148 aa, Bos taurus
MVMVLSPLLLVFILGLGLTPVAPAQDDYRYIHFLTQHYDAKPKGRNDEYCFNMMKNRRLTRPCKDRNTFIHGNKNDIKAICEDRNGQPYRGDLRISKSEFQITICKHKGGSSRPPCRYGATEDSRVIVVGCENGLPVHFDESFITPRH

SEQ ID NO: 3 - protein, 145 aa, Mus musculus
MAISPGPLFLIFVLGLVVIPPTLAQDDSRYTKFLTQHHDAKPKGRDDRYCERMMKRRSLTSPCKDVNTFIHGNKSNIKAICGANGSPYRENLRMSKSPFQVTTCKHTGGSPRPPCQYRASAGFRHVVIACENGLPVHFDESFFSL

SEQ ID NO: 4 - protein, 139 aa, Gallus gallus
MAMSSLWWTAILLLALTVSMCYGVPTYQDFLRTHVDFPKTSFPNIAAYCNVMMVRRGINVHGRCKSLNTFVHTDPRNLNTLCINQPNRALRTTQQQLPVTDCKLIRSHPTCSYTGNQFNHRVRVGCWGGLPVHLDGTFP

SEQ ID NO: 5 - protein, 125 aa, Oryctolagus cuniculus
QDDSRYKHFLTQHYDAKPFGRNDRYCETMMKRRDLTSPCKDTNTFVHGNKGSIKDVCEDKNGKPYGKNFRISKSSFQVTTCKHVGGSPWPPCRYRATSGSRNIVIACENGLPVHFDESVFQQKVH

SEQ ID NO: 6 - protein, 123 aa, Sus scrofa
KDEDRYTHFLTQHYDAKPKGRDGRYCESIMKQRGLTRPCKEVNTFIHGTRNDIKAICNDKNGEPYNNFRRSKSPFQITTCKHKGGSNRPPCGYRATAGFRTIAVACENGLPVHFDESFIITSQ

SEQ ID NO: 7 - protein, 146 aa, Equus caballus
MAMSLCPLLLVFVLGLGLTPPSLAQDDSRYRQFLTKHYDANPRGRNDRYCESMMVRRHLTTPCKDTNTFIHGSKSSIKAICGNKNGNPYGETLRISKTRFQVTTCKHAGGSPRPPCRYRATPGFRSIVIACENGLPVHFDESFFRP

SEQ ID NO: 8 - DNA, 42 bp, Artificial Sequence
  bAng0(F)NdeI oligonucleotide
ggaattccatatggctcaagatgactacagatacatacactt

SEQ ID NO: 9 - DNA, 381 bp, Bos taurus
atggctcaagatgactacagatacatacacttcctgacccagcactacgatgccaaaccaaagggccggaatgacgaatattgttttaacatgatgaaaaatcgacgcctgaccagaccttgcaaagaccgcaacacctttattcatggcaacaagaatgacattaaggccatctgtgaggacagaaatggacagccttacagaggcgatctcagaataagcaagtctgaattccagatcaccatctgcaagcataaaggaggttcctcccggcctccatgccggtacggagccacagaagactccagagtcattgttgtcggctgtgaaaatggcttgcccgtccactttgatgagtcctttatcactccacgccactag

SEQ ID NO: 10 - DNA, 47 bp, Artificial Sequence
  bAngMSH C-1 oligonucleotide
cagcggaagtgctccatggagtaggagtggcgtggagtgataaagga

SEQ ID NO: 11 - DNA, 43 bp, Artificial Sequence
  bAngMSH C-2 oligonucleotide
catggagcacttccgctggggcaagccggtgtgactcgagaat

SEQ ID NO: 12 - DNA, 25 bp, Artificial Sequence
  MSH-XhoI C-0(R) oligonucleotide
attctcgagtcacaccggcttgccc

SEQ ID NO: 13 - DNA, 429 bp, Artificial Sequence
  alpha-MSH.bAng gene
catatgtcctactccatggagcacttccgctggggcaagccggtggctcaagatgactacagatacatacacttcctgacccagcactacgatgccaaaccaaagggccggaatgacgaatattgttttaacatgatgaaaaatcgacgcctgaccagaccttgcaaagaccgcaacacctttattcatggcaacaagaatgacattaaggccatctgtgaggacagaaatggacagccttacagaggcgatctcagaataagcaagtctgaattccagatcaccatctgcaagcataaaggaggttcctcccggcctccatgccggtacggagccacagaagactccagagtcattgttgtcggctgtgaaaatggcttgcccgtccactttgatgagtcctttatcactccacgccactagctcgag

SEQ ID NO: 14 - protein, 139 aa, Artificial Sequence
  alpha-MSH.bAng protein
MSYSMEHFRWGKPVAQDDYRYIHFLTQHYDAKPKGRNDEYCFNMMKNRRLTRPCKDRNTFIHGNKNDIKAICEDRNGQPYRGDLRISKSEFQITICKHKGGSSRPPCRYGATEDSRVIVVGCENGLPVHFDESFITPRH

SEQ ID NO: 15 - DNA, 429 bp, Artificial Sequence
  bAng.alpha-MSH gene
catatggctcaagatgactacagatacatacacttcctgacccagcactacgatgccaaaccaaagggccggaatgacgaatattgttttaacatgatgaaaaatcgacgcctgaccagaccttgcaaagaccgcaacacctttattcatggcaacaagaatgacattaaggccatctgtgaggacagaaatggacagccttacagaggcgatctcagaataagcaagtctgaattccagatcaccatctgcaagcataaaggaggttcctcccggcctccatgccggtacggagccacagaagactccagagtcattgttgtcggctgtgaaaatggcttgcccgtccactttgatgagtcctttatcactccacgccactcctactccatggagcacttccgctggggcaagccggtgtgactcgag

SEQ ID NO: 16 - protein, 139 aa, Artificial Sequence
  bAng.alpha-MSH protein
MAQDDYRYIHFLTQHYDAKPKGRNDEYCFNMMKNRRLTRPCKDRNTFIHGNKNDIKAICEDRNGQPYRGDLRISKSEFQITICKHKGGSSRPPCRYGATEDSRVIVVGCENGLPVHFDESFITPRHSYSMEHFRWGKPV

(19) United States
(12) Patent Application Publication
(10) Pub. No.: US 2012/0328591 A1
(43) Pub. Date: Dec. 27, 2012

(54) METHODS FOR IMPROVING ORAL DELIVERY

(75) Inventors: Matthew McDonagh, Williamstown (AU); Angus Tester, Moonee Ponds (AU); Benjamin Cocks, Viewbank (AU); Ross Crittenden, Moonee Ponds (AU); Jianghui Wang, Bundoora (AU)

(73) Assignees: AGRICULTURE VICTORIA SERVICES PTY LTD, Attwood (AU); MURRAY GOULBURN CO-OPERATIVE CO., LTD., Brunswick (AU)

(21) Appl. No.: 13/510,167

(22) PCT Filed: Nov. 18, 2010
(86) PCT No.: PCT/US10/57295

§ 371 Date: Sep. 5, 2012

(30) Foreign Application Priority Data
Nov. 18, 2009 (AU) 2009905632

(51) Int. Cl. C12N 9/96; A61K 38/45

(52) U.S. Cl. 424/94.3; 435/188

(57) ABSTRACT

The invention provides a method of improving the oral delivery of a therapeutic agent, comprising the step of linking the therapeutic agent to a carrier protein comprising angiogenin, fusion proteins or conjugates comprising angiogenin and a therapeutic agent and their use in medicine.

The invention relates to methods for improving the oral delivery of therapeutic proteins or peptides, and in particular to methods of designing proteins or peptides with improved bioavailability when administered orally and therapeutic proteins and peptides which have improved oral bioavailability.

### BACKGROUND

Oral administration of therapeutic agents is desirable because it is generally associated with optimal compliance by the patient with the treatment regimen, and permits greater flexibility of the dosing schedule, as well as avoiding the risks, inconvenience and expense associated with administration by injection. However, the ability to utilize the oral route is limited by the ability of the drug:

(a) to be absorbed through the mucosal layers in the oral cavity, oesophagus or gut,

(b) and to survive acid and enzymic degradation in the digestive tract, and

(c) to pass across the epithelial cell layer into the systemic circulation.

Almost all pharmacological peptides are not orally available to a useful extent. In particular, hormones such as insulin, growth hormone, follicle-stimulating hormone or calcitonin, and cytokines such as interferon or interleukin, are known to have oral availabilities without special formulation well below 2%. At such levels, the temporal and inter-individual variability in availability is typically high, rendering oral administration impractical, uneconomical or dangerous.

Peptides are of increasing importance in medical treatment. However, their use has been limited by the fact that the great majority of peptides have to be administered by injection. Although alternative routes of systemic administration have been suggested, such as the pulmonary, nasal or transdermal routes, hitherto these have been developed only for a limited range of agents and suffer from limitations in tolerability and in the amount of compound that can be delivered in a single dose.

Various attempts have been made to improve the bioavailability of pharmaceuticals. These include incorporation of penetration enhancers, such as salicylates, lipid-bile salt mixes, micelles, glycerides and acylcarnitines but these are found to cause toxicity problems on most occasions.

If the pharmaceutical is a protein or peptide, attempts to improve oral bioavailability include mixing the protein or peptide with protease inhibitors such as aprotinin, soybean trypsin inhibitor and amastatin, to limit degradation of the administered therapeutic agent. Unfortunately these protease inhibitors are not selective and endogenous proteases are also inhibited by them with undesirable effects.

Other attempts to provide oral formulations of peptides have utilized protective coatings such as enteric coatings, alone or together with chemical modification of the peptide by coupling of the protein or peptide to amphiphilic oligomers or polymers comprising for example a hydrophilic polyethylene glycol moiety and a lipophilic alkyl moiety. These techniques confer very limited success. Another approach is to add an excipient that loosens the tight junctions in the gastrointestinal tract, but this approach causes tolerability problems, because the compromised barrier may admit all molecules in the vicinity, including bacteria. Also calcium alginate-coated liposome formulations have been used for colonic delivery of peptides. However, so far such approaches have found only limited application.

Some attempts to make therapeutic peptides or proteins orally bioavailable rely on the use of carrier proteins or peptides, for example vitamin B12. Such systems are still under investigation.

There is therefore a need in the art for new methods for improving the oral delivery of agents, especially proteins or peptides so that they are capable of existing in the gut intact or are capable of crossing the GI tract and entering the systemic circulation.

### SUMMARY

In a first aspect, the invention provides a method of improving the oral delivery of a therapeutic agent, comprising the step of linking the therapeutic agent to a carrier protein comprising angiogenin.

The applicant's earlier application PCT/AU2009/000602 demonstrates that angiogenin is orally available, in that it is capable of having its effect when fed to mice in their diet. Angiogenin in a milk extract has also been found to have no known toxicity at any dose, and can be effectively administered at frequencies ranging from once every few days to continuously. The inventors have now found that linking angiogenin to a therapeutic agent which is not itself orally bioavailable (or only has limited oral bioavailability) can confer substantial oral bioavailability upon that therapeutic agent.

In a second aspect, the invention provides an oral delivery system comprising angiogenin as a carrier for transporting a therapeutic agent into or across the gastrointestinal tract substantially intact

In a third aspect the invention provides a fusion protein or conjugate comprising angiogenin linked to a therapeutic agent.

In a fourth aspect, the invention provides a pharmaceutical composition, comprising a fusion protein or conjugate according to the third aspect of the invention, together with a pharmaceutically-acceptable carrier.

In one embodiment the pharmaceutical composition is for oral administration.

A fifth aspect provides use of angiogenin for linking to a therapeutic agent to improve the oral bioavailability of that therapeutic agent.

In a sixth aspect the present invention also provides methods of preventing or treating a pathological disorder in an animal in need of treatment with a therapeutic agent, by orally administering to the animal an effective amount of the fusion protein or conjugate of the third aspect of the invention or a pharmaceutical composition according to the fourth aspect of the invention, in which the therapeutic agent is not normally substantially orally bioavailable.

The seventh aspect of the present invention provides use of angiogenin in the manufacture of a medicament comprising a therapeutic agent, in which the medicament is for administering orally to a patient in need of treatment with said therapeutic agent.

It is also contemplated that angiogenin may be used to orally deliver agents for use in diagnosis and or monitoring the progression of a pathological disorder and or the effect of a further therapeutic agent on the progression of the pathological disorder.

In one embodiment the therapeutic agent is a protein or peptide.

In a further aspect the invention provides a food, neutraceutical or feed comprising the fusion protein or conjugate of the third aspect of the invention.

Without wishing to be limited by any proposed mechanism for the observed beneficial effect, it is thought that the highly folded structure of angiogenin makes it resistant to proteases present in the GI tract. Additionally it is thought that angiogenin has particular membrane binding properties and lipophilic and hydrophilic balance which enhance its transport (and the transport of anything to which it is conjugated) across the mucosal layers in the gastrointestinal (GI) tract. Such properties are also proposed to enable angiogenin and anything to which it is conjugated to transport across the epithelial cell membrane.

### FIGURES

In the non-limiting examples that follow the invention is described with reference to the attached figures in which:

FIG. 1 shows digestion resistance of native bAng over 2 hours of digestion with pepsin at pH 3.0 at an enzyme to substrate ratio of 1:20. Molecular weight is given in lane 1 with profile and intensity of bAng given in lanes 4 to 12 for native bAng digested with pepsin.

FIG. 2 shows a map of the N-terminal vector construct of αMSH.bAng constructed into pET30C vector using Nde I and Xho I restriction site.

FIG. 3 shows a map of the C-terminal vector construct of αAng.aMSH constructed into pET30C vector using Nde I and Xho I restriction sites.

FIG. 4 provides primer sequences for the construction of αMSH.bAng and bAng.aMSH constructed into pET30C vector using Nde I and Xho I restriction sites.

FIG. 5 provides a. DNA sequence and protein sequence for α-MSH-bAng from the pET30C vector construct and b. DNA sequence and protein sequence for bAng-α-MSH from the pET30C vector construct.

FIG. 6 shows SDS polyacrylamide gels of purified αMSH.bAng and bAng-α-MSH fusion proteins expressed in E. coli from constructs within pET30C vector.

FIG. 7 shows the proportion of radio-labelled αMSH.bAng fusion protein in the blood (a.) and tissues (b.) of C57Black/6J mice expressed as a percentage of total administered by oral gavage.

FIG. 8 shows the proportion of radio-labelled bAng.αMSH fusion protein in the blood (a.) and tissues (b.) of C57Black/6J mice expressed as a percentage of total administered by oral gavage.

### DETAILED DESCRIPTION

The inventor proposes that the balance of hydrophobic and hydrophilic residues provided by angiogenin allows it to protect therapeutic agents with which it is administered from acid and enzymic degradation in the digestive tract and to allow the therapeutic agent to be absorbed through the mucosal layers in the oral cavity, oesophagus or gut, and optionally to pass across the epithelial cell layer into the systemic circulation, substantially intact. By “substantially intact” we mean without removing the therapeutic activity of agent.

Use of the term “therapeutic agent” in this specification includes a drug (e.g., a small molecule drug, e.g., an antibiotic), a medicine, a detectable label, a protein (e.g., an enzyme), protein-based compound (e.g., a protein complex comprising one or polypeptide chain) and a polypeptide (peptide). The agent is not limited in its therapeutic use.

As referred to herein “oral delivery” or “oral administration” are intended to encompass any administration or delivery to the GI tract and includes administration directly to the oropharyngeal cavity, and administration via the mouth in which the actual absorption of the peptide or polypeptide into the gut or systemic circulation takes place in the gastrointestinal tract, including the stomach, small intestine, or large intestine. Oral administration as used herein encompasses sublingual administration (administration by application under the tongue of the recipient, representing one form of administration via the oropharyngeal cavity) and buccal administration (administration of a dosage form between the teeth and the cheek of the recipient).

Oral delivery and oral administration may be used interchangeably herein.

Bioavailability as used herein refers to the availability of the therapeutic agent in the gut, bloodstream or systemic circulation.

It is preferred that the transporting activity which is effected by the carrier does not affect the integrity of the GI tract. The transporting of a therapeutic agent may result, for example, in the delivery of the agent to the systemic circulation of an individual.

The term “conjugate” is intended to mean a combination of a carrier and a therapeutic agent that is not the carrier. The conjugation may be chemical in nature, such as via a linker, or genetic in nature for example by recombinant genetic technology, such as in a fusion protein with for example a reporter molecule (e.g. green fluorescent protein, β-galactosidase, Histag, etc.).

The conjugates and pharmaceutical compositions of the invention may additionally be administered with or combined with other compounds to provide an operative combination or combination therapy. It is intended to include any chemically compatible combination of pharmaceutically-active agents, as long as the combination does not eliminate the activity of the conjugate of the invention.

When the conjugates of this invention are administered in combination therapy with other agents, they may be administered sequentially or concurrently to an individual. Alternatively, pharmaceutical compositions according to the present invention may be comprised of a combination of a carrier-agent conjugate of the present invention in association with a pharmaceutically acceptable excipient, as described herein, and another therapeutic or prophylactic agent known in the art.

The therapeutic agent for oral delivery may be used to treat any disease or disorder.

The term “therapeutic agent” or “agent” is intended to mean an agent and/or medicine and/or drug used to treat the symptoms of a disease or condition, injury or infection and includes, but is not limited to, antibiotics, antimicrobial agents, anti-cancer agents and anti-angiogenic agents.

The term “condition” is intended to mean any situation causing pain, discomfort, sickness, disease or disability (mental or physical) to or in an individual.

Examples of a protein or protein-based therapeutic agents which may be delivered using the angiogenin carrier or conjugated with angiogenin for delivery into or across the GI tract encompassed herein includes, without limitation, an antibody, an antibody fragment (e.g., an antibody binding fragment such as Fv fragment, F(ab)₂, F(ab)₂′ and Fab and the like), a peptidic- or protein-based drug (e.g., a positive pharmacological modulator (agonist) or an pharmacological inhibitor (antagonist)) etc. Other examples of agent which are encompassed herein include cellular toxins (e.g., monomethyl auristatin E (MMAE), toxins from bacteria endotoxins and exotoxins; diphtheria toxins, botunilum toxins, tetanus toxins, perussis toxins, staphylococcus enterotoxins, toxin shock syndrome toxin TSST-1, adenylate cyclase toxin, shiga toxin, cholera enterotoxin, and others) and anti-angiogenic compounds (endostatin, catechins, nutraceuticals, chemokine IP-10, inhibitors of matrix metalloproteinase (MMPIs), anastellin, vironectin, antithrombin, tyrosine kinase inhibitors, VEGF inhibitors, antibodies against receptors, Herceptin®, Avastin® and panitumumab and others), IFNα, β or γ, G-CSF, TPO, EPO, PtHR, antimicrobials like angiogenin, cathelicidins, GPCR peptide ligands, human growth hormone, antiinflammatory peptides including BPI and lipocortins, cytokines including interleukin 10, 4 and 13, gut hormones including secretin, gastrin, cholecystokinin, VIP, GIP, motilin and enteroglucagon and synthetic peptides designed to activate receptors (peptide mimetics).

Particularly relevant for transport into the gut from the gut lumen (particularly across the gut mucosa) and not necessarily completely across gut epithelia into the bloodstream are therapeutic agents for gut health and immunity. Such therapeutic agents can be used to enhance gut tissue function. They include angiogenin, particularly optimised for gut enhancing or antimicrobial activity, RNase 5, RNase 4, antimicrobial agents, anti-inflammatory peptides including synthetics, BPI and lipocortins, cytokines including interleukin 10, 4 and 13, gut hormones including secretin, gastrin, cholecystokinin, VIP, GIP, motilin and enteroglucagon and any agents identified as blocking immune response in coelic disease or irritable bowel syndrome.

Also in accordance with the present invention, the agent may be a small molecule drug such as an anticancer drug. An anticancer drug encompassed by the present invention may include, for example, a drug having a group allowing it's conjugation to the carrier of the present invention. Examples of anticancer drug includes, for example, without limitation, a drug which may be selected from the group consisting of paclitaxel (Taxol), docetaxel, vinblastine, vincristine, etoposide, doxorubicin, cyclophosphamide, taxotere, melphalan, chlorambucil, and any combination, Leptin may be used for treatment of obsesity.

The expression “small molecule drug” is intended to mean a drug having a molecular weight of 1000 g/mol or less.

It is to be understood herein that when more than one carrier conjugation site are available or present, more than one agent may be conjugated to the carrier of the present invention. Therefore, the conjugate may comprise one or more agents. The conjugate may be active by itself, i.e., the agent may be active even when associated with the carrier. Also in accordance with the present invention, the compound may or may not be released from the carrier i.e., generally after transport into or across the GI tract. The compound may therefore be releasable from the conjugate (or from the carrier) and may become active thereafter. More particularly, the agent may be releasable from the carrier after transport into or across the GI tract.

The present invention also relates, in a further aspect to a method for treating a mammal (e.g., a patient) in need thereof comprising administering a conjugate and/or a pharmaceutical composition of the present invention to the mammal.

### Angiogenin

The angiogenin used in the present invention may be from any species but particularly includes from human, bovine, porcine, equine, avian, ovine, rat, chicken, turkey or mouse angiogenin. The angiogenin may comprise SEQ ID NO: 1 (human), SEQ ID NO: 2 (bovine), SEQ ID NO: 3 (mouse), SEQ ID NO: 4 (chicken), SEQ ID NO: 5 (rabbit), SEQ ID NO: 6 (pig), SEQ ID NO: 7 (horse), or any other sequence encoding angiogenin or a functional fragment thereof capable of inducing growth of myoblasts in cell culture.

```
(SEQ ID NO: 1)
                10 20 30 40 50 60
MVMGLGVLLL VFVLGLGLTP PTLAQDNSRY THFLTQHYDA KPQGRDDRYC ESIMRRRGLT
                70 80 90 100 110 120
SPCKDINTFI HGNKRSIKAI CENKNGNPHR ENLRISKSSF QVTTCKLHGG SPWPPCQYRA
              130 140
TAGFRNVVVA CENGLPVHLD QSIFRRP
(SEQ ID NO: 2)
                10 20 30 40 50 60
MVMVLSPLLL VFILGLGLTP VAPAQDDYRY IHFLTQHYDA KPKGRNDEYC FNMMKNRRLT
                70 80 90 100 110 120
RPCKDRNTFI HGNKNDIKAI CEDRNGQPYR GDLRISKSEF QITICKHKGG SSRPPCRYGA
              130 140
TEDSRVIVVG CENGLPVHFD ESFITPRH
(SEQ ID NO: 3)
                10 20 30 40 50 60
MAISPGPLFL IFVLGLVVIP PTLAQDDSRY TKFLTQHHDA KPKGRDDRYC ERMMKRRSLT
                70 80 90 100 110 120
SPCKDVNTFI HGNKSNIKAI CGANGSPYRE NLRMSKSPFQ VTTCKHTGGS PRPPCQYRAS
              130 140
AGFRHVVIAC ENGLPVHFDE SFFSL
(SEQ ID NO: 4)
                10 20 30 40 50 60
MAMSSLWWTA ILLLALTVSM CYGVPTYQDF LRTHVDFPKT SFPNIAAYCN VMMVRRGINV
                70 80 90 100 110 120
HGRCKSLNTF VHTDPRNLNT LCINQPNRAL RTTQQQLPVT DCKLIRSHPT CSYTGNQFNH
              130
RVRVGCWGGL PVHLDGTFP
(SEQ ID NO: 5)
                10 20 30 40 50 60
QDDSRYKHFL TQHYDAKPFG RNDRYCETMM KRRDLTSPCK DTNTFVHGNK GSIKDVCEDK
                70 80 90 100 110 120
NGKPYGKNFR ISKSSFQVTT CKHVGGSPWP PCRYRATSGS RNIVIACENG LPVHFDESVF
QQKVH
(SEQ ID NO: 6)
                10 20 30 40 50 60
KDEDRYTHFL TQHYDAKPKG RDGRYCESIM KQRGLTRPCK EVNTFIHGTR NDIKAICNDK
                70 80 90 100 110 120
NGEPYNNFRR SKSPFQITTC KHKGGSNRPP CGYRATAGFR TIAVACENGL PVHFDESFII
TSQ
(SEQ ID NO: 7)
                10 20 30 40 50 60
MAMSLCPLLL VFVLGLGLTP PSLAQDDSRY RQFLTKHYDA NPRGRNDRYC ESMMVRRHLT
                70 80 90 100 110 120
TPCKDTNTFI HGSKSSIKAI CGNKNGNPYG ETLRISKTRF QVTTCKHAGG SPRPPCRYRA
              130 140
TPGFRSIVIA CENGLPVHFD ESFFRP
```

The angiogenin can include one or more conservative amino acid substitutions compared to the amino acid sequence of a known angiogenin. Non-limiting examples of conservative amino acid substitutions are Phe/Tyr; Ala/Val; Leu/Ile; Arg/His; Ser/Thr; etc. The angiogenin can also include insertions or deletions (including truncations) of one or more amino acid residues, compared to the amino acid sequence of a known angiogenin. Further, the angiogenin can include one or more naturally occurring polymorphisms. The angiogenin can be completely or partially synthetic. An angiogenin can also be a consensus sequence, derived, e.g., by comparing the angiogenin coding sequences from two or more species, and deriving therefrom a consensus sequence, using standard methods. An optimised angiogenin sequence can also be used, for example a sequence that includes mutations that confer greater activity, more protease resistance, etc. A particularly optimised angiogenin sequence is one in which RNase activity is reduced or prevented.

Particular fragments and variants include one or more conserved domains such as sequences encoding a catalytic core or a cell binding site. By a “catalytic core” is meant an internal region of the polypeptide excluding signal peptide and N- and C-terminal variable regions.

Two distinct regions of angiogenin are required for its angiogenic activity including a catalytic site containing His-13, Lys-41, and His-115 that is capable of cleaving RNA and a noncatalytic, cell binding site encompassing minimally residues 60-68. RNase activity and receptor binding capacity, while required, are not sufficient for angiogenic activity: endocytosis and nuclear translocation are required as well.

Activity may be increased or decreased by changing key amino acids at or near the active site with improved activity substituting Asp-116 to His being an example. Functional studies indicate Arg-5 and Arg-33 are also important for activity.

Increasing protease and heat stability of RNases is possible, so RNase5/angiogenin stability and protease resistance can be improved which may assist in delivery of the conjugate or pharmaceutical composition.

Cellular uptake of angiogenin in proliferating endothelial cells is mediated by domains and is not dependent upon RNase activity as enzymatically inactive mutants can be internalized. K41Q and H13A mutants for example are enzymatically inactive but are translocated. Improved versions of angiogenin more readily internalised by cells and more potent are within the scope of the present invention, and such variants can be tested for by conducting in vitro uptake and activity tests on epithelial and muscle cells in culture.

For cellular transport angiogenin receptor binding and endocytosis will likely need to be retained or enhanced. Variants with enhanced gut and cell delivery function can be tested and screened for in vitro in caco-2 intestinal epithelial cell systems.

Improved versions of angiogenin that can more readily internalised by cells and more potent can be envisaged, and such variants can be tested for by conducting in vitro uptake and activity tests on epithelial and muscle cells in culture, with testing in mice.

Any known angiogenin can be used in accordance with the invention, including angiogenin from mouse, human, cow, sheep, etc.

A suitable amino acid sequence for angiogenin generally has at least about 70%, at least about 80%, at least about 85%, at least about 90%, at least about 95%, or at least about 98%, or higher, amino acid sequence identity with a known sequence for angiogenin. Sequence similarity is calculated based on a reference sequence, which may be a subset of a larger sequence, such as a conserved motif. Algorithms for sequence analysis are known in the art, such as BLAST, described in Altschul et al. (1990), J. Mol. Biol. 215:403-10 (using default settings).

Also suitable are angiogenin sequences encoded by nucleic acid molecules that hybridize under stringent hybridization conditions to a known angiogenin coding sequence. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. For example, high stringency conditions include aqueous hybridization (e.g., free of formamide) in 6×SSC (where 20×SSC contains 3.0 M NaCl and 0.3 M sodium citrate), 1% sodium dodecyl sulfate (SDS) at 65° C. for about 8 hours (or more), followed by one or more washes in 0.2×SSC, 0.1% SDS at 65° C. For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature.

### Therapeutic Agent

A therapeutic agent as used herein is a therapeutic agent which has no or limited oral bioavailability.

Preferably the therapeutic agent is sufficiently stable in the GI tract but has difficulty in crossing the gut mucosa or entering the bloodstream. Alternatively, the therapeutic protein or peptide is protected in the GI tract by encapsulation, enteric coating or the like. Ideally such protection is maintained until the angiogenin and therapeutic protein or peptide reaches the lower intestine, where angiogenin is able to actively or passively transport the therapeutic agent across the gut mucosa and optionally across the gut epithelium where it is released into the bloodstream.

Sufficiently stable as used herein refers at least 20% of the administered agent remaining after 30 minutes of exposure in the GI tract.

In one embodiment the therapeutic protein or peptide is a peptide of 20 or fewer amino acids, potentially providing substantial oral activity when administered using the oral delivery system of the present invention. Examples of such therapeutic peptides include α-melanocyte stimulating hormone, vasopressin, oxytocin, enkephalin, somatostatin and conotoxins including ACV1.

In one embodiment the therapeutic protein or peptide is a peptide of between 21 and 40 amino acids, for example parathyroid hormone (PTH 1-34) as described in the examples. Other examples of such therapeutic proteins or peptides include glucagon-like peptide (GLP-1), calcitonin, PYY3-36, oxyntomodulin, Gastric Inhibitory Peptide (GIP), endorphin, and related members of the superfamily.

In one embodiment the therapeutic protein or peptide is a peptide of between 41 and 60 amino acids. Examples of such therapeutic peptides are insulin and Insulin Like Growth Factor-1 (IGF-I).

In one embodiment the therapeutic protein or peptide is a peptide of between 61 and 80 amino acids.

In one embodiment the therapeutic protein or peptide is greater than 80 amino acids. Possible examples of such therapeutic proteins or peptides include growth hormone, interleukins, or other large growth factors.

Key molecules for delivery by the method of the invention include interferon α and β, G-CSF, TPO, EPO, PtHR, antimicrobials like cathelicidins, GPCR peptide ligands, hGH and synthetic peptides designed to activate receptors (peptide mimetics) and gut enhancing agents as described above.

### Linkage

To improve the oral bioavailability of the therapeutic agent it must be linked to angiogenin. The linkage may be a covalent or non-covalent linkage. Persons skilled in the art would appreciate appropriate linkers which retain the function of the therapeutic agent and the ability of angiogenin to cross the GI tract.

In one embodiment, angiogenin is attached to the therapeutic agent by the N terminus of angiogenin. In another embodiment angiogenin is linked to the therapeutic agent by the C-terminus of angiogenin. If the agent is a protein or peptide it may be attached to angiogenin via its N or C terminus.

The angiogenin and the therapeutic agent may be linked by any convenient method which confers bioactivity to the therapeutic agent. As angiogenin is relatively large (over 100 amino acids) it is preferably synthesised using recombinant methods or isolated from milk or plasma, and subsequently isolated and linked to the therapeutic agent using enzymic methods, or the whole conjugate or fusion protein may be synthesized using recombinant methods. Suitable methods will be well known to those skilled in the art, and the most convenient method for any given situation can be readily selected.

The preferred linkage site may vary, depending on the nature of the therapeutic agent. In one particular embodiment the conjugate comprises the N-terminus of the angiogenin linked to the C-terminus of the therapeutic protein or peptide, but this will depend in the main on which addition point preserves bioactivity.

In one embodiment the angiogenin and therapeutic agent are separated by a spacer, such as polyglycine. The use of a spacer may prevent steric hindrance and subsequent reduction in activity of the therapeutic agent.

The spacer or linker may include a cleavage site for enzymes present in gut epithelium or proteases between the angiogenin and therapeutic agent so that the angiogenin is cleaved from the therapeutic agent once it has entered the mucosal layer or crossed the GI tract and is in the bloodstream, so as to prevent any inhibition of the activity of the therapeutic agent due to the presence of angiogenin.

Adding chemical modifications to the protein such as protective groups on the end of the fusion protein would provide protease resistance eg adding a synthesized a conjugate incorporating a D-amino acid at the C or N terminus or other aminoacid modifications at the C and N termini.

### Conjugate

A conjugate as defined herein comprises angiogenin as defined herein linked to a therapeutic agent as defined herein, in any order.

The angiogenin may be conjugated to one or more therapeutic agents, which may be the same or different. For conjugation of a plurality of therapeutic agents a polylysine linker may be used.

### Pharmaceutical Compositions

An aspect of the invention provides various pharmaceutical compositions useful for preventing or treating pathological conditions. The pharmaceutical compositions according to one embodiment of the invention are prepared by bringing a fusion protein or conjugate according to the third aspect of the invention, or an analogue, derivative or salt thereof, into a form suitable for administration to a subject, using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Other pharmaceutically acceptable carriers include non-toxic excipients, including salts, preservatives, buffers and the like, as described in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference.

Preservatives include antimicrobials, anti-oxidants, and chelating agents. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions of the invention may benefit from encapsulation or an enteric coating to reduce degradation in the GI tract.

The pharmaceutical compositions according to the invention may be administered in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects.

Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions are also suitable for oral use, and normally contain the active materials in admixture with excipients suitable for the manufacture of aqueous suspensions, for example saline. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be
(a) a naturally occurring phosphatide such as lecithin;
(b) a condensation product of an alkylene oxide with a fatty acid, for example, polyoxyethylene stearate;
(c) a condensation product of ethylene oxide with a long chain aliphatic alcohol, for example, heptadecaethylenoxycetanol;
(d) a condensation product of ethylene oxide with a partial ester derived from a fatty acid and hexitol such as polyoxyethylene sorbitol monooleate, or
(e) a condensation product of ethylene oxide with a partial ester derived from fatty acids and hexitol anhydrides, for example polyoxyethylene sorbitan monooleate.

Dosage levels of the conjugate of the present invention will vary widely depending on the potency of the conjugate, usually be of the order of about 1 μg to about 5 mg per kilogram body weight, from about 100 μg to about 500 mg per patient per day). The amount of active ingredient which may be combined with the carrier materials to produce a single dosage will vary, depending upon the host to be treated and the particular mode of administration. For example, a formulation intended for oral administration to humans may contain about 100 μg to 500 mg of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

### Foods and Neutraceuticals

The fusion protein or conjugate can be administered in a food composition, for example a functional food, or animal feed. Such foods are suitable for consumption by any individual. As used herein, the term “individual” includes human and non-human individuals. Non-human individuals include animals, particularly mammals, e.g., farm animals such as cows, pigs, sheep, goats and poultry, pets and companion animals such as horses, cats, dogs, guinea pigs, rats and mice, and aquatic animals such as fish and animals used for aquaculture etc.

The term “nutraceutical formulation” refers to a food or part of a food that offers medical and/or health benefits including prevention or treatment of disease.

Nutraceutical products range from isolated nutrients, dietary supplements and diets, to genetically engineered designer foods, functional foods, herbal products and processed foods such as cereal, soup and beverages. The term “functional foods,” refers to foods that include “any modified food or food ingredients that may provide a health benefit beyond the traditional nutrients it contains.”

Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; and the like.

A subject food product or nutraceutical formulation includes the fusion protein or conjugate of the third aspect and at least one additional food-grade component. Suitable components include, but are not limited to, mono- and disaccharides; carbohydrates; proteins; amino acids; fatty acids; lipids; stabilizers; preservatives; flavoring agents; coloring agents; sweeteners; antioxidants, chelators, and carriers; texturants; nutrients; pH adjusters; emulsifiers; stabilizers; milk base solids; edible fibers; and the like. The food component can be isolated from a natural source, or can be synthesized. All components are food-grade components fit for human consumption.

Examples of suitable monosaccharides include sorbitol, mannitol, erythrose, threose, ribose, arabinose, xylose, ribulose, glucose, galactose, mannose, fructose, and sorbose. Non-limiting examples of suitable disaccharides include sucrose, maltose, lactitol, maltitol, maltulose, and lactose.

Suitable carbohydrates include oligosaccharides, polysaccharides, and/or carbohydrate derivatives. As used herein, the term “oligosaccharide” refers to a digestible linear molecule having from 3 to 9 monosaccharide units, wherein the units are covalently connected via glycosidic bonds. As used herein, the term “polysaccharide” refers to a digestible (i.e., capable of metabolism by the human body) macromolecule having greater than 9 monosaccharide units, wherein the units are covalently connected via glycosidic bonds. The polysaccharides may be linear chains or branched. Carbohydrate derivatives, such as a polyhydric alcohol (e.g., glycerol), may also be utilized as a complex carbohydrate herein. As used herein, the term “digestible” in the context of carbohydrates refers to carbohydrate that are capable of metabolism by enzymes produced by the human body. Examples of polysaccharides that are non-digestible carbohydrates are cellulose, resistant starches (e.g., raw corn starches) and retrograded amyloses (e.g., high amylose corn starches). Non-limiting examples carbohydrates include raffinoses, stachyoses, maltotrioses, maltotetraoses, glycogens, amyloses, amylopectins, polydextroses, and maltodextrins.

Suitable fats include, but are not limited to, triglycerides, including short-chain (C₂-C₄) and long-chain triglycerides (C₁₆-C₂₂).

Suitable texturants (also referred to as soluble fibers) include, but are not limited to, pectin (high ester, low ester); carrageenan; alginate (e.g., alginic acid, sodium alginate, potassium alginate, calcium alginate); guar gum; locust bean gum; psyllium; xanthan gum; gum arabic; fructo-oligosaccharides; inulin; agar; and functional blends of two or more of the foregoing.

Suitable emulsifiers include, but are not limited to, propylene glycol monostearate (PGMS), sodium stearoyl lactylate (SSL), calcium stearoyl lactylate (CSL), monoglycerides, diglycerides, monodiglycerides, polyglycerol esters, lactic acid esters, polysorbate, sucrose esters, etc.

Edible fibers include polysaccharides, oligosaccharides, lignin and associated plant substances. Suitable edible fibers include, but are not limited to, sugar beet fiber, apple fiber, pea fiber, wheat fiber, oat fiber, barley fiber, rye fiber, rice fiber, potato fiber, tomato fiber, other plant non-starch polysaccharide fiber, and combinations thereof.

Suitable flavoring agents include natural and synthetic flavors, “brown flavorings” (e.g., coffee, tea); dairy flavorings; fruit flavors; vanilla flavoring; essences; extracts; oleoresins; juice and drink concentrates; flavor building blocks (e.g., delta lactones, ketones); and the like; and combinations of such flavors. Examples of botanic flavors include, for example, tea (e.g., preferably black and green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardamom, nutmeg, cassia bark, buchu, cinnamon, jasmine, haw, chrysanthemum, water chestnut, sugar cane, lychee, bamboo shoots, vanilla, coffee, and the like.

Suitable sweeteners include, but are not limited to, alitame; dextrose; fructose; lactilol; polydextrose; xylitol; xylose; aspartame, saccharine, cyclamates, acesulfame K, L-aspartyl-L-phenylalanine lower alkyl ester sweeteners, L-aspartyl-D-alanine amides; L-aspartyl-D-serine amides; L-aspartyl-hydroxymethyl alkane amide sweeteners; L-aspartyl-1-hydroxyethylalkane amide sweeteners; and the like.

Suitable anti-oxidants include, but are not limited to, tocopherols (natural, synthetic); ascorbyl palmitate; gallates; butylated hydroxyanisole (BHA); butylated hydroxytoluene (BHT); tert-butyl hydroquinone (TBHQ); and the like.

Suitable nutrients include vitamins and minerals, including, but not limited to, niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iron, zinc, copper, calcium, phosphorous, iodine, chromium, molybdenum, and fluoride.

Suitable coloring agents include, but are not limited to, FD&C dyes (e.g., yellow #5, blue #2, red #40), FD&C lakes; Riboflavin; β-carotene; natural coloring agents, including, for example, fruit, vegetable, and/or plant extracts such as grape, black currant, aronia, carrot, beetroot, red cabbage, and hibiscus.

Exemplary preservatives include sorbate, benzoate, and polyphosphate preservatives.

Suitable emulsifiers include, but are not limited to, diglycerides; monoglycerides; acetic acid esters of mono- and diglycerides; diacetyl tartaric acid esters of mono- and diglycerides; citric acid esters of mono- and diglycerides; lactic acid esters of mono- and diglycerides; fatty acids; polyglycerol esters of fatty acids; propylene glycol esters of fatty acids; sorbitan monostearates; sorbitan tristearates; sodium stearoyl lactylates; calcium stearoyl lactylates; and the like.

Suitable agents for pH adjustment include organic as well as inorganic edible acids. The acids can be present in their undissociated form or, alternatively, as their respective salts, for example, potassium or sodium hydrogen phosphate, potassium or sodium dihydrogen phosphate salts. Exemplary acids are edible organic acids which include citric acid, malic acid, fumaric acid, adipic acid, phosphoric acid, gluconic acid, tartaric acid, ascorbic acid, acetic acid, phosphoric acid and mixtures thereof.

The fusion protein or conjugate may be present in the food product/nutraceutical formulation in an amount of from about 0.01% to about 50% by weight, e.g., from about 0.01% to about 0.1%, from about 0.1% to about 0.5%, from about 0.5% to about 1.0%, from about 1.0% to about 2.0%, from about 2.0% to about 5%, from about 5% to about 7%, from about 7% to about 10%, from about 10% to about 15%, from about 15% to about 20%, from about 20% to about 25%, from about 25% to about 30%, from about 30% to about 35%, from about 35% to about 40%, from about 40% to about 45%, or from about 45% to about 50% by weight.

Where the food product is a beverage, the food product generally contains, by volume, more than about 50% water, e.g., from about 50% to about 60%, from about 60% to about 95% water, e.g., from about 60% to about 70%, from about 70% to about 80%, from about 80% to about 90%, or from about 90% to about 95% water.

Where the food product is a bar, the food product generally contains, by volume, less than about 15% water, e.g., from about 2% to about 5%, from about 5% to about 7%, from about 7% to about 10%, from about 10% to about 12%, or from about 12% to about 15% water.

In some embodiments, the food product/nutraceutical is essentially dry, e.g., comprises less than about 5%, water.

Monosaccharides, disaccharides, and complex carbohydrates, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each. Soluble fibers, edible fibers, and emulsifiers, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each.

Other components discussed above, if present, are present in amounts ranging from about 0.001% to about 5% by weight of the composition.

### Methods of Treatment

The fusion protein, conjugate, pharmaceutical, food, neutraceutical or feed compositions of the present invention may be used in methods of treatment of any pathological disorder which may be treated by the therapeutic agent, in which the therapeutic agent is administered orally.

Reference herein to treatment is intended to encompass prevention of the pathological disorder or alleviation of the pathological disorder.

The pathological disorder to be treated by the present invention may be any disorder which is treated by the therapeutic agent.

In the description of the invention and in the claims which follow, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used herein, the singular forms “a”, “an”, and “the” include the corresponding plural reference unless the context clearly dictates otherwise. Thus, for example, a reference to “a peptide” includes a plurality of such peptides, and a reference to “an amino acid” is a reference to one or more amino acids.

Where a range of values is expressed, it will be clearly understood that this range encompasses the upper and lower limits of the range, and all values in between these limits.

Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are described.

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

Unless otherwise indicated, the present invention employs conventional chemistry, protein chemistry, molecular biological and enzymological techniques within the capacity of those skilled in the art. Such techniques are well known to the skilled worker, and are explained fully in the literature.

The invention will now be described in detail by way of reference only to the following non-limiting examples and figures.

### Example 1

### Testing Protease Resistance of Recombinant Bovine Angiogenin

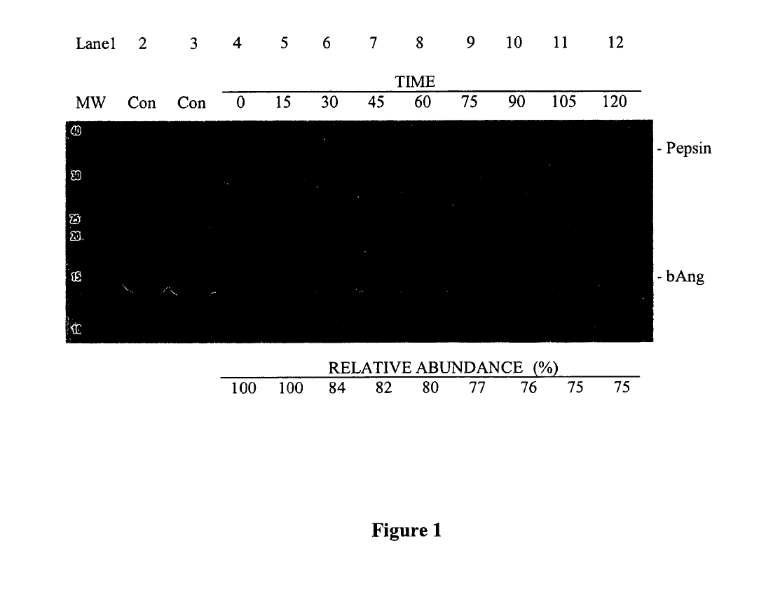

A 30 ug aliquots of bAng was prepared for digestion with pepsin (porcine stomach mucosa Type A; Sigma 900-75-6). Digestion was conducted in enzyme/substrate ratios of 1:20. and the trial was controlled under constant temperature of 37° C. and a constant pH of 3. Samples were taken every 15 minutes for a total digestion time of 120 minutes. Each sample was snap frozen at −80° C. immediately to cease the digestion process. The level of intact bAng after pepsin digestion was resolved using SDS PAGE. Gels were stained using comassie blue and the band densities were quantified using Odyssey imaging system V3.0. FIG. 1 demonstrates the digestion of native bAng to pepsin. Quantitation of band intensities in FIG. 1 demonstrate that 75% of bAng protein is present as intact non-degraded protein following 2 hours of digestion in conditions simulating the stomach.

### Example 2

### Production of Angiogenin Fusion Proteins Using Recombinant Bacteria

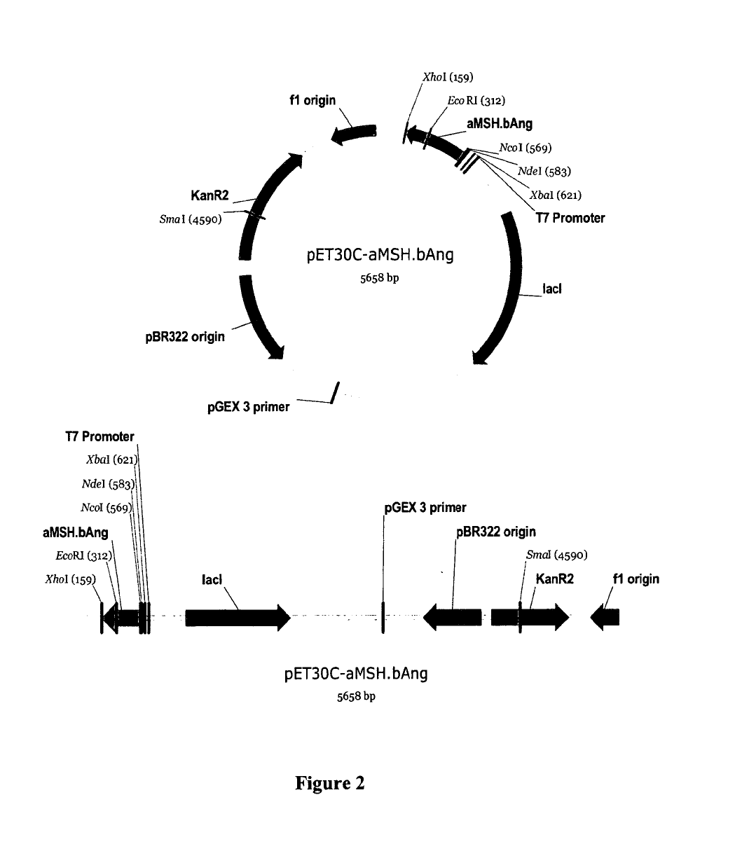

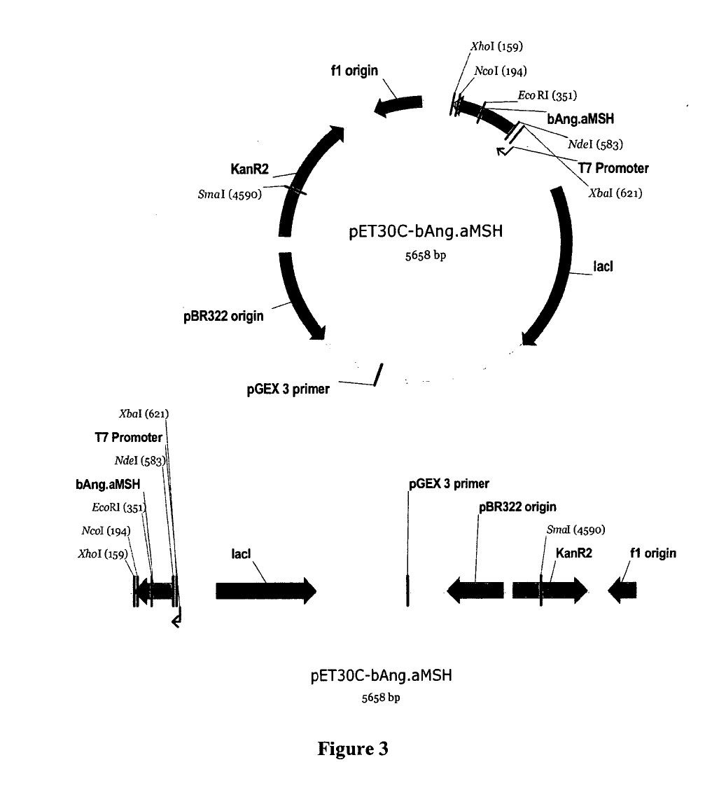

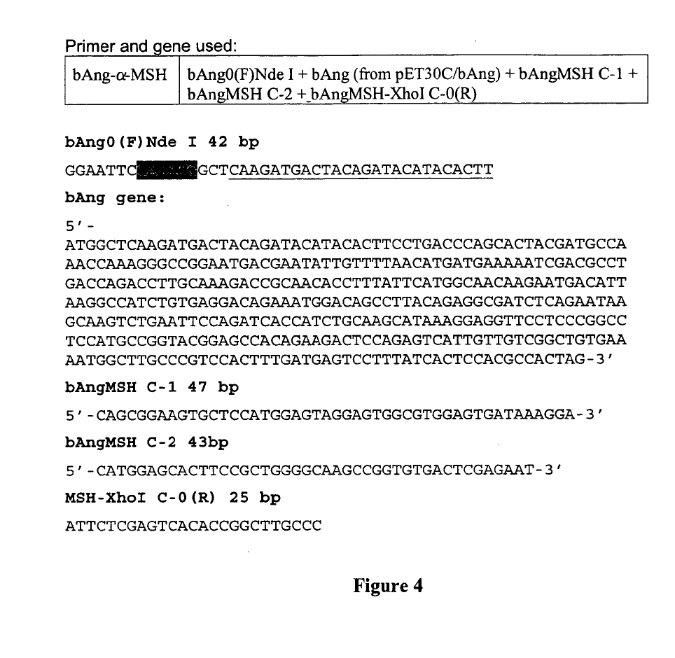

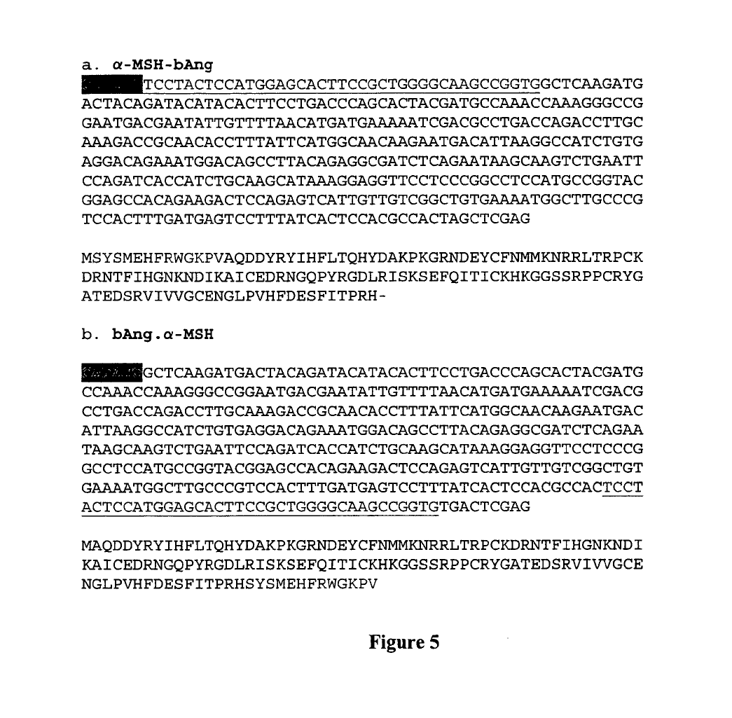

Production of a N- and C-terminal αMSH.bAng constructs was conducted using pET30C vector and the constructs were assembled and amplified by PCR, inducing Nde I and Xho I restriction sites and αMSH (see FIGS. 2 and 3). The product was cloned into the same sites of pET30C vector after digestion of Nde I and Xho I. The primer sequences are given (FIG. 4) and the construct sequences was confirmed by DNA sequencing (FIGS. 5a. and 5b.).

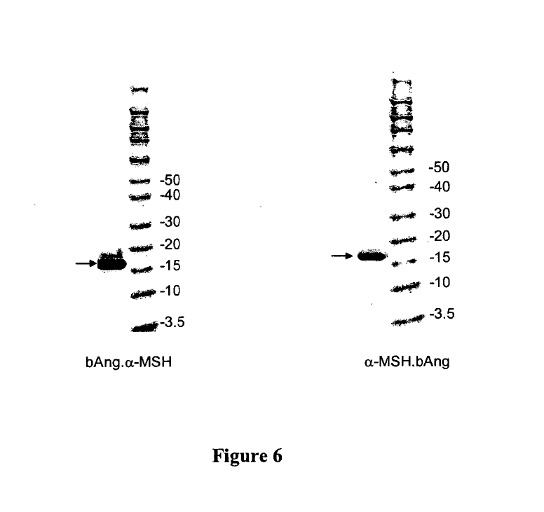

E. coli strain BL21(DE3)pLysS, carrying pET30C/bAng.α-MSH and pET30C/α-MSH.bAng, was used for expression of rbAng fusion proteins. The isolated inclusion body was dissolved in denaturing buffer containing 6 M guanidine hydrochloride (GdnHCl), 100 mM Tris/HCl (pH 8), 1 mM EDTA, 100 mM NaCl, 10 mM DTT at the protein concentration about 5 mg/ml. The denaturing solution was then slowly diluted to refolding buffer to 0.2 mg protein ml-1, 0.5 mM DTT, 0.3 M GdnHCl with 100 mM Tris/HCl (pH 8), 1 mM EDTA, 0.3 mM GSSG, 1.5 mM GSH. The solution was then incubated without stirring in a vessel opened to the air at 4° C. for 72 h. After refolding was completed, the solution was concentrated by ultrafiltration and dialysed against milliQ water at 4° C. and then loaded onto Pierce strong cation Exhange mini spin column following manufacture instruction. The rbAng fusion proteins were eluted by 1 M NaCl. The purified recombinant proteins are shown in FIG. 6.

### Example 3

### Radio-Labelled N- and C-Terminal Angiogenin Fusion Proteins are Absorbed into Bloodstream Within 15 Minutes of Oral Gavage in Mice

A total of 200 μg of both bAng.α-MSH and αMSH.bAng was purified using the protocols outlined in Example 2 above. Using a chloramine-T, sodium metabisulfite (CT/SMB) iodination, an aliquot of 20 ug of each fusion protein was labelled with 1.0 mCi of I125. Free or un-incorporated ¹²⁵I was separated from labelled bAng.α-MSH and αMSH.bAng using HPLC with a PD10 protein column. The incorporation rate for ¹²⁵I was 79% for both proteins such that a total of 790 μCi of labelled bAng.α-MSH and αMSH.bAng constructs was available for oral gavage in mice. The total labelled bAng.α-MSH and αMSH.bAng were diluted individually to 3.0 ml prior to gavage of individual animals.

A total of Thirty male, 8 week old, C57Black/6J mice were used in the experiment. Mice were weighed at 11:00 am and then allocated to post-gavage sampling at 15, 30, 45, 60, 120 or 180 min (n=2 or 3 mice for bAng.α-MSH and αMSH.bAng at each time point). Each animal was dosed via oral gavage with 200 μl of appropriate radio labelled solution (equivalent to 53 μCi) and sacrificed 15, 30, 45, 60, 120 or 180 min by cervical dislocation.

Blood was collected from the heart via cardiac puncture and placed in heparinised tubes on ice, then stored at −20° C. The brain was removed and weighed before being snap frozen in liquid nitrogen. The heart, liver, kidney and quadriceps muscle were quickly excised before being weighed and snap frozen by clamping and placed in liquid nitrogen. Excised tissues were then stored at −80° C.

For analysis of radioactivity in tissues approximately 40 mg of tissue was placed into a 2 ml eppendorf tube containing 300 μl of ice cold RIPA buffer (50 mM Tris-HCl ph7.4, 150 mM NaCl, 1 mM EDTA, 1% NP-40, 25% Sodium deoxycholate, 1 μg/ml Pics 1, 1 μg/ml Pics 2, 2 mM PMSF, 1 mM Na pyrophosphate, 10 mM NaF, 1 mM NaVO4). The tissue was then homogenised using a hand held Pro 200 homogeniser (Pro Scientific Inc, Oxford, Conn., USA) for approximately 10 sec or until there were no visible lumps of tissue remaining. After homogenisation the samples were stored frozen in liquid nitrogen and then placed on ice to thaw before analysis. Once thawed the samples were vortexed vigorously for 5 secs then spun in a centrifuge at 4° C., 3000 g for 30 sec. Samples were then counted for 1 min in 100 μl duplicates on a gamma counter (Cobra II, auto-gamma, Packard Bioscience Company).

Protein concentration of each homogenised sample was analysed by Pierce BCA protein assay (Thermo Scientific, Rockford, Ill., USA). Samples were diluted 1 in 40 in RIPA buffer for the assay.

Blood samples were analysed by diluting 1 in 30 in MQ water before mixing well by vortexing. 100 μl of the sample was then added in duplicate to tubes for counting as described above.

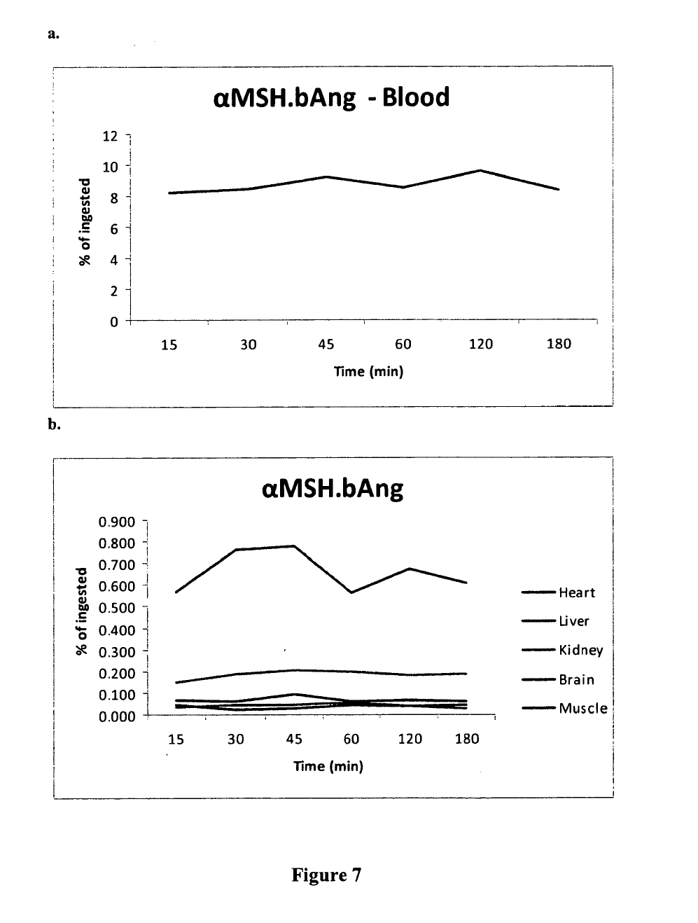

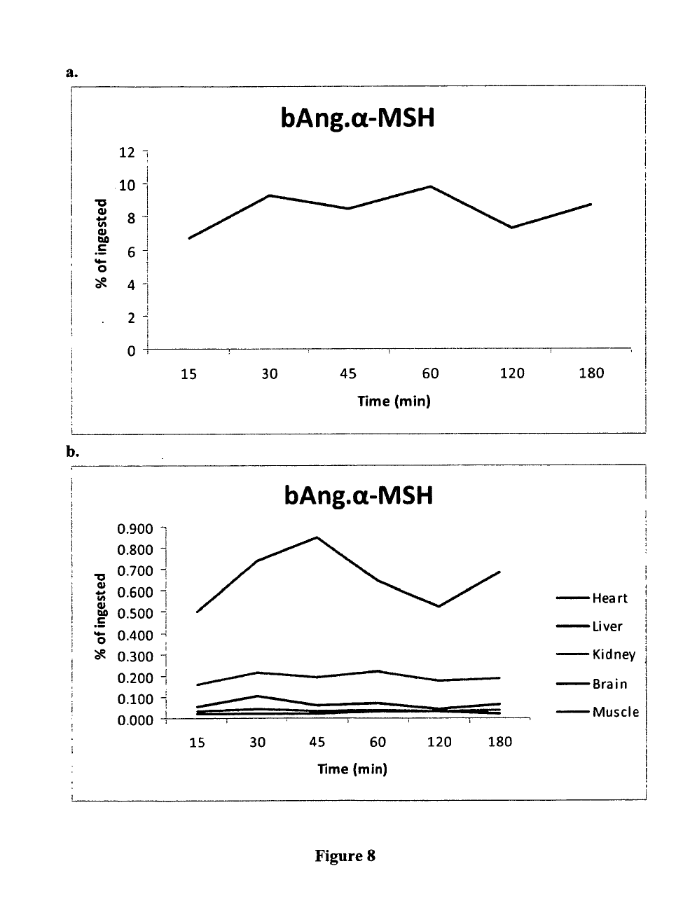

FIG. 7.a. demonstrates that in excess of 8% of the ingested αMSH.bAng was present within the blood of C57Black/6J mice within 15 minutes of oral gavage. This level was maintained across the course of the experiment. A similar uptake of uptake of bAng.αMSH into blood was observed (FIG. 8.a.). Relatively lower levels were seen in the liver (approximately 0.6% of total ingested) and kidney (approximately 0.2% of total ingested) for both αMSH.bAng (FIG. 7.b.) and bAng.αMSH (FIG. 8.b.). Uptake into the quadriceps muscle, heart and brain tissue were all less than 0.1% of total ingested αMSH.bAng and bAng.αMSH.

The rapid absorption of at least 8% of both fusion proteins within 15 minutes of oral gavage and the maintenance of this level over 3 hours is atypical for digested proteins. Free amino acids and small di or tri peptides absorbed across the gastrointestinal tract following protein digestion show a more gradual increase in absorption and distinct peak in concentration within this 3 hour sampling period reflecting digestion and absorption. Free amino acids are also cleared rapidly from the blood by incorporation into tissues including skin, muscle, kidney, liver, heart and brain tissue. The accumulation of a low percentage of label in the liver and kidney is expected due to high metabolic activity in these tissues and non-specific uptake out of the blood. A lack of incorporation into skeletal muscle, heart and brain clearly demonstrates that the label present in the blood is not free amino acid and is intact protein.

Mansanès et. al. (2001) demonstrate in rats that 80% of amino acids absorbed into the portal bloodstream within the first hour following gavage are sequestered into tissues including skeletal muscle, kidney, brain and liver. In mice, more than 90% of amino acids from hydrolysed protein sources are absorbed across the gut within the first 6 hours post-gavage (Oesser et. al., 1999) and are sequestered into skin, liver, kidney, spleen, cartilage and skeletal muscle from the circulation.

R. M. Masanès, I. Rafecas and X. Remesar (2001) Absorption of a Protein Gavage in Zucker Lean Rats. Influence of Protein Content in the Diet. Archives of Physiology and Biochemistry 109: 168-174.

S. Oesser, M. Adam, W, Babel and J. Seifert (1999) Oral Administration of 14C Labeled Gelatin Hydrolysate Leads to an Accumulation of Radioactivity in Cartilage of Mice (C57/BL). Journal of Nutrition 129: 1891-1895,

1. A method of improving the oral delivery of a therapeutic agent, comprising the step of linking the therapeutic agent to a carrier comprising angiogenin.

2. An oral delivery system comprising angiogenin as a carrier for transporting a therapeutic agent into or across the gastrointestinal tract.

3. A fusion protein or conjugate comprising angiogenin linked to a therapeutic agent.

4. A pharmaceutical composition comprising the fusion protein or conjugate of claim 3 and a pharmaceutically-acceptable carrier.

5. The pharmaceutical composition of claim 4 for oral administration.

6. (canceled)

7. A method of preventing or treating a pathological disorder in an animal in need of treatment with a therapeutic agent, by orally administering to the animal an effective amount of the fusion protein or conjugate of claim 3, in which the therapeutic agent is not normally substantially orally bioavailable.

8. The conjugate of claim 3 in which the therapeutic agent is a protein or peptide.

9. The method of claim 1 in which the therapeutic agent is a protein or peptide.

10. The delivery system of claim 2 in which the therapeutic agent is a protein or peptide.

11. The pharmaceutical composition of claim 8 in which the therapeutic agent is a protein or peptide.

12. The conjugate of claim 8 in which the therapeutic protein or peptide has 20 or fewer amino acids.

13. The conjugate of claim 8 in which the therapeutic protein or peptide has 21 to 40 amino acids.

14. The conjugate of claim 8 in which the therapeutic protein or peptide has 41 to 60 amino acids.

15. The conjugate of claim 8 in which the therapeutic protein or peptide has 61 to 80 amino acids.

16. The conjugate of claim 8 in which the therapeutic protein or peptide has 81 or more amino acids.

17. The fusion protein or conjugate of claim 3 comprising angiogenin of SEQ ID NO: 1, 2, 3, 4, 5, 6 or 7 linked to a therapeutic agent.

18. The conjugate of claim 17 in which the therapeutic agent comprises an anti-microbial peptide.

19. The conjugate of claim 17 in which the therapeutic agent comprises parathyroid hormone.

20. The conjugate of claim 17 in which the therapeutic agent comprises insulin.

21. The conjugate of claim 17 in which the therapeutic agent comprises interferon α or β, G-CSF, TPO, EPO, PtHR, antimicrobials, angiogenin, anti-inflammatory agents, cathelicidins, GPCR peptide ligands, hGH or synthetic peptides designed to activate receptors (peptide mimetics).

22. A method of preventing or treating a pathological disorder in an animal in need of treatment with a therapeutic agent, by orally administering to the animal an effective amount of the pharmaceutical composition of claim 4, in which the therapeutic agent is not normally substantially orally bioavailable.
